(19) United States
(12) Patent Application Publication
(10) Pub. No.: US 2026/0348942 A1
(43) Pub. Date: Nov. 5, 2026

(54) STIMULUS RESPONSIVE WIRELESS SENSOR INTEGRATED SMART URINE BAG

(71) Applicant: Purdue Research Foundation, West Lafayette, IN (US)

(72) Inventors: Rahim Rahimi, West Lafayette, IN (US); Muhammad Masud Rana, West Lafayette, IN (US); Sarath Gopalakrishnan, Santa Rosa, CA (US); Akshay Krishnakumar, West Lafayette, IN (US)

(73) Assignee: Purdue Research Foundation, West Lafayette, IN (US)

(21) Appl. No.: 19/664,741

(22) Filed: Apr. 30, 2026

Related U.S. Application Data
(60) Provisional application No. 63/797,425, filed on Apr. 30, 2025.

(51) Int. Cl. G01N 33/493; A61F 5/44; G01N 27/22; H01Q 1/22

(52) U.S. Cl. CPC G01N 33/493; A61F 5/4404; G01N 27/227; H01Q 1/22

(57) ABSTRACT

A system may include a urine bag and an inductor-capacitor (LC) resonator-based sensor disposed in the urine bag. The LC resonator-based sensor may include an antenna connected to a capacitor with a stimuli-responsive coating disposed on the capacitor. The stimuli-responsive coating may dissolve in response to presence of a stimuli in the urine thereby changing the capacitance of the capacitor and altering the resonance characteristics of the LC resonator-based sensor.

### CROSS-REFERENCE TO RELATED APPLICATIONS

This application claims the benefit of U.S. Provisional Patent Application No. 63/797,425, filed Apr. 30, 2025, the entire contents of which are incorporated herein by reference in their entirety.

### TECHNICAL FIELD

This disclosure relates to biomarker detection and, in particular, to biomarker detection in urine.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates an example of the Smart Urinary Bag (SUB) system.

FIG. 2 illustrates an example of a sensor platform.

FIG. 3 illustrates an example of manufacturing a sensor platform.

FIG. 4 illustrates an example of a wireless reader device for interrogating one or more LC resonator-based sensor tags.

### DETAILED DESCRIPTION

According to the World Health Organization, 421 million patients are hospitalized annually, with 13-20 million admitted to intensive care units (ICUs). Among ICU patients, around 75% require urinary catheters due to immobilization or incontinence, facilitating urine evacuation and improving comfort. However, urinary catheterization carries a high risk of hospital-associated infections, as it provides a pathway for bacteria to enter the urinary tract. Patients with catheters face a 3-10% daily risk of developing catheter-associated urinary tract infections (CAUTIs), potentially leading to serious complications such as pyelonephritis, renal failure, and sepsis. Early detection is critical, as timely intervention can prevent CAUTIs from advancing to severe outcomes requiring prolonged antibiotic treatment.

Currently, the gold standard for UTI detection is laboratory-based urine culture, which is highly sensitive and specific but labor-intensive, requiring skilled personnel and up to two days for analysis. This delay may allow infections to progress. Rapid screening methods, such as observing changes in urine color, odor, and volume, provide general information but lack the specificity needed to accurately detect infection biomarkers, limiting their effectiveness for early UTI diagnosis and prognosis.

Clinical biomarkers such as pH, glucose, nitrite, xanthine oxidase (XOD), myeloperoxidase (MPO), and leukocyte esterase play a vital role in assessing infection levels and understanding the severity of UTIs. Common approaches for monitoring biomarkers such as pH, nitrite, or blood levels in urine include colorimetric dipstick techniques that utilize chemical strips sensitive to specific stimuli. Enzyme-based colorimetric assays, like ELISA, are also used to detect levels of XOD and MPO in urine. However, these techniques require repeated sample collection from the urine bag, which increases the risk of biofluid contamination due to direct contact and handling. The need for frequent sampling also limits these methods' applicability for continuous, real-time monitoring and poses significant exposure risks for healthcare technicians.

Efforts to minimize sample handling and enable wireless monitoring of urine biomarkers have primarily focused on adapting conventional colorimetric assays with optical readout systems for wireless data collection and storage. For instance, Catherwood et al. and Seo et al. have developed systems that use colorimetric assays with optical readouts to measure nitrite levels, a known UTI biomarker. In their work, Catherwood et al. paired a traditional colorimetric urine dipstick with a wireless LoRa system for transmitting data to a central unit, allowing for nitrite detection in a small urine sample applied to a test strip. However, this approach does not support real-time monitoring or integration within a standard urinary bag.

Similarly, Seo et al. developed a partially autonomous system by integrating a colorimetric sensor with a miniaturized optical detection device for wireless monitoring in a diaper. Although this method allows for initial measurements upon first contact with urine, it does not enable continuous monitoring and is not suitable for urinary bag integration. Due to the single-use nature of colorimetric assays, which complete the analysis upon initial urine exposure, these systems are inherently limited to discrete measurements rather than continues monitoring.

To address these limitations and provide continuous wireless monitoring of urine in patients with indwelling catheters, our group previously developed a contactless Sticker-Type Antenna for Remote Sensing (STARS) platform for early UTI detection in critically ill ICU patients. STARS is a multifunctional, contactless, and non-invasive system that remotely monitors urine flow rate and conductivity in catheterized patients. Although STARS demonstrated promising results in controlled laboratory conditions, it lacks the selectivity needed to detect specific infection biomarkers essential for UTI identification. Therefore, there is a critical need for a sensor platform that can provide continuous, remote assessment of key UTI biomarkers directly within the urinary bag, offering an efficient and safer alternative for UTI monitoring in ICU settings.

Among various infection-related biomarkers, urine pH is a commonly known indicator of UTI progression in ICU patients. A meta-analysis of 18 studies revealed a strong correlation between the increase in urine pH and the progression of uropathogenic infections. Hence, various efforts have been made to develop electrochemical probe-based sensors to monitor fluctuations of pH in urine. While these sensors can be used for real-time continuous monitoring, they can be laborious and expensive and require frequent calibration with standard buffer solutions, limiting their applicability and accuracy in real-time scenarios. Furthermore, as the infected urine typically exceeds a pH of 7.5, it is adequate to employ a pH sensor with a narrow range (pH 5~9) that can detect the increase in urine alkalinity. However, this correlation is not definitive, as diet and other factors can influence urine pH, making it an unreliable standalone indicator for UTI diagnosis.

Another potential biomarker gaining attention in UTI detection is XOD. Recent studies have shown that elevated levels of XOD (>483 U/L) in urine strongly correlate with bacterial infection progression. While the reasons for this association remain unclear, it presents an intriguing avenue for further exploration. The limitations of each biomarker in UTI risk detection in CAUTIs necessitate complementary biomarker detection for a more accurate evaluation of urine conditions. Most studies of biomarkers are based on colorimetric assays, which are sparse measurements requiring laboratory equipment and settings, lacking real-time and continuous patient monitoring for early detection of any signs of infection.

As an alternative, various electrochemical sensors, such as potentiometric and amperometric types, with integrated wireless communication technologies namely RFID, Bluetooth, and ZigBee have been developed. These offer the potential for remote monitoring of urine conditions within the bag to detect electrochemical fluctuations and assess UTIs. However, despite reports of their use for monitoring various physiological parameters like wounds, sweat, and saliva, they face significant challenges. A typical wireless sensing setup in such systems requires a chemical sensing device, power source, data acquisition device, and a wireless communication device. Integrating these sensors with complex electronics like microcontrollers and wireless devices increases the overall device complexity and cost. While efforts have been made to reduce the complexity of such systems and onboard batteries with near-field communication (NFC) tags with cellphone readers for their portability, these still necessitate integrated electronics and application-specific integrated circuits (ASICs) for data communication. This increases sensor cost and hinders their disposability which is a crucial factor for integration into urine bags. Additionally, the manufacturing processes for these sensors often rely on time-consuming and expensive photolithography techniques, limiting their scalability for disposable applications.

To address these deficiencies and other problems in the state of the art, a Smart Urinary Bag (SUB) system is disclosed. The system may include an integrated sensor platform designed for real-time monitoring of UTI-associated biomarkers in urine directly within the urinary bag. The sensor platform incorporates two interdigitated electrode (IDE) patterns coated with stimuli-responsive coatings to selectively detect elevations biomarkers. By way of example, the stimuli-responsive coating may be responsive to pH and xanthine oxidase (XOD) levels-two critical biomarkers for urinary infections. These IDEs connect to antenna forming sensor tags. By way of example, the antennas may include inductor spiral coils that enable battery-free, wireless monitoring of urine conditions through near-field inductive coupling with an external reader. The sensor tags, measuring 10 cm×6 cm, and the coating thickness were optimized using High-Frequency Structure Simulator (HFSS) software to ensure effective wireless readability and sensitivity for practical miniaturization and integration into standard urinary bags. Various experimentation provided systematic characterization of the functional coatings and confirmed effective compositions that allowed reliable wireless detection of elevated pH (>8) and XOD (>500 U/L) levels in urine, with a response time of less than 3 hours. The sensors maintained stable performance over 14 days under physiologically relevant conditions with high specificity, as verified through tests with both artificial and real urine. In one experiment, a fully integrated system was tested under simulated patient conditions with varying pH and XOD levels. The platform successfully identified high-risk UTI conditions, enabling timely alerts to healthcare providers for immediate intervention. Among the many technical advancements provided by this technology, the system may enhance patient monitoring in ICUs by reducing healthcare provider burden, facilitating early identification of UTI risks, and promoting timely interventions, ultimately reducing antibiotic overuse and addressing the challenge of rising antibiotic-resistant bacterial strains.

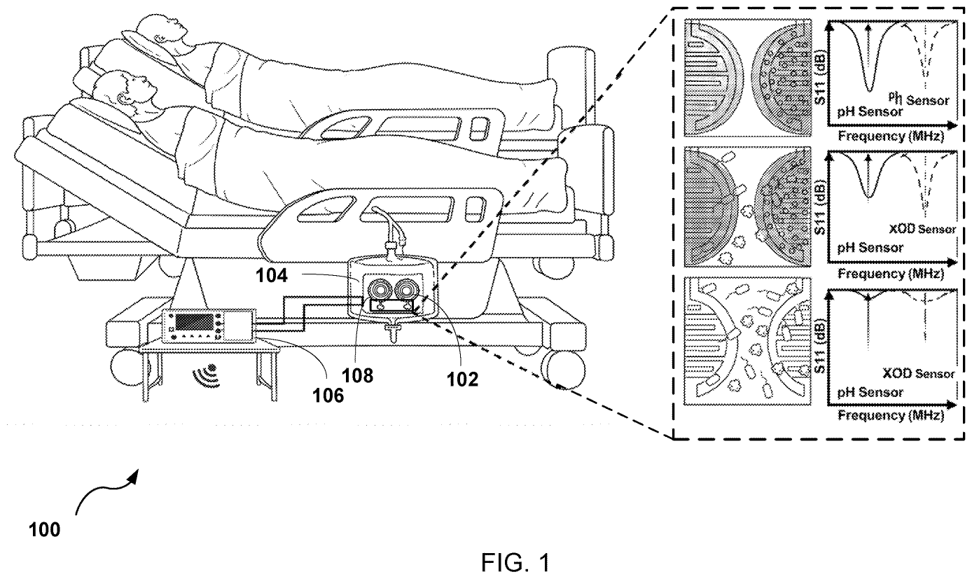

FIG. 1 illustrates an example of the Smart Urinary Bag (SUB) system. The system 100 may include a disposable sensor platform 102 included in or integrated with a urine bag 104. The sensor platform 102 includes at least one LC resonator-based sensor and a reusable, portable reader device for non-contact, remote interrogation of the sensor.

The sensor platform may be placed inside the urine bag 104, with the functionalized coated electrodes positioned at the bottom of the bag. For example, the sensor platform may be a flexible sheet which can be rolled up and placed in the opening of a standard urine bag. Alternatively, the sensor platform may be attached to an inside wall of the urine bag during manufacturing. In any event, the IDE of the sensor platform may disposed along the bottom of the bag. This orientation ensures immediate contact with the collected urine, enabling rapid screening of pH and XOD levels (or any other suitable biomarker).

At high biomarker levels, the coating on the electrodes dissolves, resulting in reduced capacitance and altered resonance characteristics of the LC resonator circuit. The resulting change in resonance characteristics is wirelessly transmitted from the spiral coil, which is inductively coupled with the reader antenna for data communication.

The system may include a wireless reader device 106. The wireless reader device 106 may include a reader antenna 108 which may be positioned proximate to the sensor platform 102. An electromagnetic signal is transmitted from the reader antenna 108 to power the inductive antenna of the LC resonator circuit, and the reflected signal is received. The reflected signal obtained using a swept-frequency Vector Network Analyzer (VNA), records two distinct reflection coefficient (S₁₁(fᵣ)) characteristics corresponding to the pH and XOD spiral coils. When pH and XOD levels exceed the designated thresholds, the functionalized polymer dissolves, leading to an electrical short in the IDE capacitors and the disappearance of the corresponding peak in the reflection coefficient (S₁₁(fᵣ)). The sensor tags within the urine bag are designed to operate effectively over 14 days, during which healthcare providers typically empty the bag daily.

For optimal operation, the urine bag should remain in the standard ICU configuration, hanging upright from the side of the bed. To minimize cross-contamination risks, the outlet of the emptying port should be kept off the floor and cleaned frequently with ethanol. This approach helps ensure that contamination is minimized, supporting reliable long-term monitoring within ICU environments.

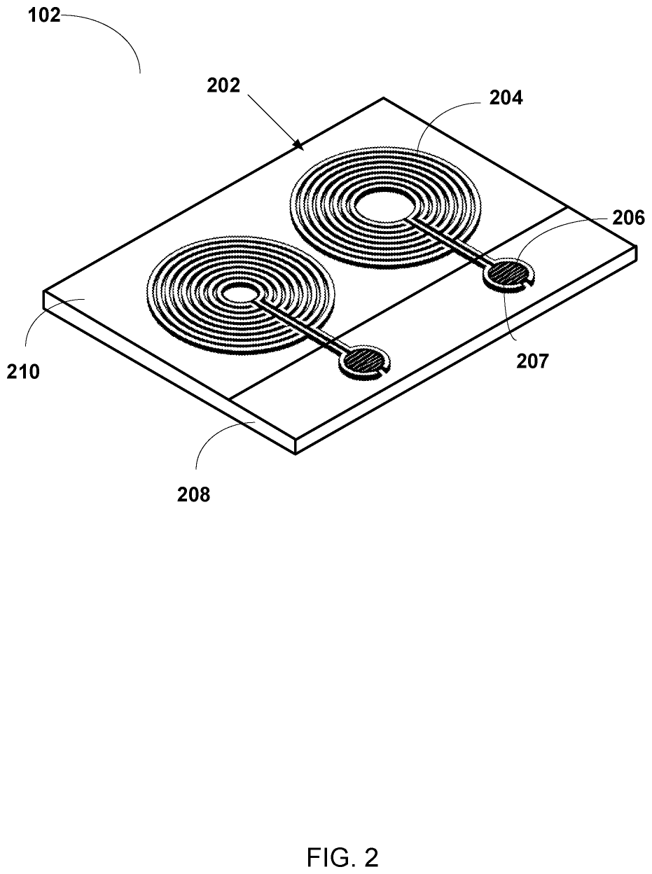

FIG. 2 illustrates an example of the sensor platform 102. The sensor platform 102 may include an inductor-capacitor (LC) resonator sensor 202. The LC resonator sensor (herein also referred to as the LC tank circuit, LC resonator circuit or LC resonator sensor) may include an antenna 204. The antenna 204 may include an such as a spiral inductor. The LC Resonator sensor 202 may further include a capacitor 206 connected to the antenna. The capacitor 206 may include electrodes on which a stimulus responsive coating 207 may be applied. For example, the capacitor 206 may be interdigitated electrodes (IDE). The IDEs may be coated with a stimulus responsive material, such as a functionalized polymer. The coating may dissolve in response to the presence of target concentration of biomarker in the urine, thereby changing the capacitance of the capacitor and altering the resonance characteristics of the LC resonator sensor 202. In various examples described herein, polymer coatings responsive to pH and XOD were used, though other biomarkers may be targeted.

The stimuli-responsive coating 207 may be applied across the interdigitated electrodes. When biomarker levels exceed threshold values indicative of infection risk, the stimulus-responsive coating on the IDEs dissolves, causing the IDE electrodes to short-circuit. This results in the elimination of the resonance peak in the reflection coefficient (S₁₁) at the resonant frequency (fᵣ), signaling elevated risk conditions. This change in the resonance signature is wirelessly detected by the external reader coils, which are aligned with the sensor tag and attached externally to the urine bag.

The antenna 204 may include an inductor. For example, the inductor may include a coiled inductor. One technical advantage to using the coiled inductor in combination with the IDE is that the two components can be connected without crossing wires or leads. Also, the number of windings on the coiled inductor may be varied to change the resonant characteristics of the antenna 204.

In some examples, the sensor platform 102 may include multiple LC resonator sensors. For example, FIG. 2 shows two LC resonator sensors, but the sensor platform may have more (or even just one). The stimuli-responsive coating on each of the LC resonator sensors may be responsive to a different stimuli, respectively. For example, a first stimuli-responsive coating may be applied to a first capacitor and a second stimuli responsive coating may be applied to a second capacitor. The antenna of each of the LC resonator sensors have a different resonant characteristics that are detectable by a remote device which interrogates the LC resonator sensor, thereby allowing differentiation among the LC resonator sensors.

The sensor platform may include a substrate 208. The LC resonator-based sensor(s) may be located on a surface of the substrate 208. The substrate 208 may include a material on which the LC resonator-based sensor(s) are formed. For example, the substrate may include PET or some other suitable non-conductive material. In some examples, the substrate may be flexible to allow the sensor platform to be rolled or manipulated into a urine bag. They substrate may include, in some examples, an adhesive on at least one side in order to stick to a urine bag.

In some examples, a passivation layer 210 may cover the antenna(s) and leave the capacitor(s) exposed. In doing so, the passivation layer 210 may protect the antenna(s) from any corrosive or interfering effects of the urine, leaving only the capacitor(s) and stimuli-responsive coating(s) exposed to the urine. The passivation layer may include, for example, polyurethanes, silicone, or any other barrier type coating of film that can be glued onto the surface. In some examples, the passivation layer 210 may include an adhesive on an outer surface in order to secure the sensor platform 102 to a urine bag.

The sensor platform 102 may be integrated with a urine bag. In some examples, the sensor platform may be added to the urine bag. For example, the sensor platform may be rolled up and inserted into an opening of the urine bag. Alternatively, the sensor platform may be attached to or directly printed on an inside wall of the urine bag.

Various embodiments of the sensor platform 102 are possible. In one example, the sensor platform 120 may include two interdigitated electrodes coated with functional polymers that are specifically tuned for the detection of elevated pH and XOD levels. These IDEs are connected to two spiral coils, forming a pair of LC resonators. The sensor's reflection coefficient (S₁₁) characteristics, which are remotely interrogated via a two-coil reader system, depend on the capacitance and inductance of the IDEs and spiral coils, respectively. Upon exposure to high biomarker concentrations in the urine, the stimuli-responsive coatings on the IDEs dissolve, leading to a short circuit in the capacitive element of the LC resonator and the loss of its S₁₁, which indicates a high-risk condition marked by elevated pH or XOD levels.

The sensor manufacturing process may leverage recent advancements in scalable, flexible, low-cost sensor fabrication, including laser processing on metalized plastic substrates and automated dispensing of stimuli-responsive polymeric coatings. Selective laser processing of metalized plastic films enables the use of cost-effective materials, such as aluminum-coated polyethylene terephthalate (PET), commonly used in standard packaging, for constructing the LC tank. This approach makes sensor fabrication more scalable and economically viable for disposable applications. Furthermore, selective functionalization of the LC tank sensors with stimuli-responsive polymers allows for rapid customization to detect various analytes of interest within the urine bag. The non-contact design of the reusable reader device enhances system practicality, enabling continuous monitoring by positioning the reader near the urine bag for remote interrogation. By facilitating continuous, non-invasive monitoring of physiological pH and XOD levels, this technology aims to enable early UTI detection, reducing complications and improving patient outcomes through timely medical interventions. This early detection capability can help prevent infection progression, minimize antibiotic overuse, and lower the risk of severe conditions such as sepsis.

To maximize scalability and cost-effectiveness, disposable sensor tags may be fabricated on commercially available flexible metallized PET sheets using a simple laser processing technique. This one-step laser-etching process allows precise patterning of aluminum (Al) traces on the PET sheet, forming a twin spiral coil connected to IDEs for simultaneous pH and XOD detection.

The IDE sensor tags are coated with stimulus-responsive polymers using a standard drop-casting method, imparting selectivity for specific urinary biomarkers. The coated IDEs, in conjunction with the connected spiral coil, function as a stimulus-responsive LC resonator with two distinct resonant frequencies, each dedicated to detecting pH and XOD levels.

### Stimuli Responsive pH and XOD Polymeric Coatings

To enhance specificity in detecting elevated pH and XOD levels in urine, stimuli-responsive polymers were applied to the IDE electrodes on the sensor tag. These polymers—either cationic or anionic—were designed to selectively dissolve under specific pH conditions, enabling the sensor to respond to changes in the surrounding environment.

For pH detection, an anionic polymer containing acidic carboxyl (—COOH) groups was coated onto the IDE electrodes. In acidic environments, these carboxyl groups remain protonated, minimizing ionization and keeping the polymer in an insoluble matrix. Under alkaline conditions, however, deprotonation of these carboxyl groups occurs, forming negatively charged —COO⁻ ions as interactions with OH-ions in the surrounding solution take place. When exposed to elevated pH levels in the urine bag, the polymer coating dissolves, which causes the IDE electrode to shift from a capacitive to a conductive (short-circuit) state. This shift leads to a detectable change in the resonant peak of the LC resonator, allowing for real-time monitoring of pH increases associated with heightened UTI risk.

For XOD detection, a specialized polymer composite was developed to dissolve in response to XOD's enzymatic activity. This specificity was achieved through the catalytic reaction between XOD and hypoxanthine (HX). When XOD catalyzes the oxidation of HX to uric acid, a localized pH decrease occurs. This mechanism was used as an indirect approach to detect XOD by triggering a pH drop. The IDE electrode for XOD sensing was functionalized with HX-infused filter paper and coated with a cationic polymer composite engineered to dissolve in acidic environments below pH 5. In such conditions, protonation of the amine (—NH₂) groups in the polymer takes place, forming —NH₃⁺ ions. The increase in electrostatic repulsion causes the polymer to swell and dissolve. At elevated XOD levels, this localized pH decrease activates the dissolution of the cationic polymer, which short-circuits the IDE and results in a detectable shift in the resonant characteristics of the LC resonator, monitored externally by the reader coil.

To ensure the pH and XOD polymer coatings met specificity and activation requirements within physiologically relevant conditions associated with UTI risk, various polymer compositions and mixtures were systematically characterized. This process enabled the identification of optimal formulations that achieve threshold-based dissolution within the pH and XOD ranges that correlate with UTI risk levels, thereby providing accurate and reliable detection of infection indicators.

Additional biomarker detection is possible. For example, several additional urinary biomarkers that are clinically relevant for detecting and monitoring infections, inflammation, kidney function, and metabolic disorders. Many of these biomarkers can be detected using stimuli-responsive polymer coatings, which respond to changes in pH, enzymatic activity, oxidative stress, or specific molecular interactions. Below are some example biomarkers along with possible stimuli-responsive sensing mechanisms that could be integrated into the platform, as shown in Table 1.

TABLE 1
Stimuli Response Coating Examples

| | | | Example | |
|---|---|---|---|---|
| Analyte/ | Clinical | Sensing | Materials/ | Potential |
| Biomarker | Relevance | Mechanism | Approaches | Use/Output |
| Myeloperoxidase | Neutrophil-derived | Oxidation- | Thiol- or | Detection of |
| (MPO) | enzyme; | responsive polymers | phenol-containing | acute |
| | elevated in UTIs | degrade or | polymers; | inflammatory |
| | and inflammatory | change | fluorinated or | responses; |
| | conditions | conductivity in | methacrylate- | correlation |
| | | response to | based polymers | with |
| | | MPO-mediated | engineered to | leukocyte |
| | | oxidative stress | dissolve under | activity |
| | | (e.g., | specific redox | |
| | | hypochlorous | states | |
| | | acid, HOCl) | | |
| Urea | Primary | Urease | Urease | Indirect urea |
| | nitrogenous | catalyzes | immobilized in | detection via |
| | waste in urine; | hydrolysis of | pH-responsive | pH-triggered |
| | abnormal levels | urea to | hydrogels or | polymer |
| | indicate renal | ammonia, | polymer | swelling or |
| | dysfunction or | increasing local | matrices | dissolution |
| | dehydration | pH and | | |
| | | triggering | | |
| | | changes in | | |
| | | pH-sensitive | | |
| | | hydrogels | | |
| Nitrite (NO₂⁻) | Classic UTI | Diazotization- | Azo dye | Detection of |
| | marker produced | sensitive polymers | precursors; | bacterially |
| | by | undergo | nitrite-reactive | mediated |
| | nitrate-reducing | structural, | electrochemical | UTIs via |
| | bacteria (e.g., E. | dielectric, or | or conversion | nitrite |
| | coli) | colorimetric | polymers | presence |
| | | changes in | | |
| | | response to | | |
| | | nitrite | | |
| Creatinine | Key indicator of | Selective | MIPs formed | Quantitative |
| | kidney function | binding to | using functional | or threshold- |
| | with stable daily | molecularly | monomers | based |
| | production | imprinted | such as | assessment |
| | | polymers (MIPs) | acrylamide or | of renal |
| | | causes | methacrylic | function |
| | | dielectric or | acid | |
| | | conductivity | | |
| | | changes | | |
| Protein/ | Early indicator of | Protein-binding | Carboxylated | Detection of |
| Albumin | kidney damage, | polymers swell | or sulfonated | early kidney |
| (Microalbuminuria) | especially in | or dissolve upon | hydrogels; | damage via |
| | diabetic | protein | hydrophilic | LC signal |
| | nephropathy | interaction | PEG-based | attenuation |
| | | | polymer | |
| | | | systems | |
| Glucose | Elevated in | Glucose | GOx | Indirect |
| | diabetes and | oxidase (GOx) | embedded in | glucose |
| | kidney disorders | oxidizes glucose | pH-sensitive | detection via |
| | | to gluconic acid, | polymer | pH-triggered |
| | | causing a | matrices | polymer |
| | | localized pH | | dissolution |
| | | decrease | | and |
| | | | | resonance |
| | | | | shift |

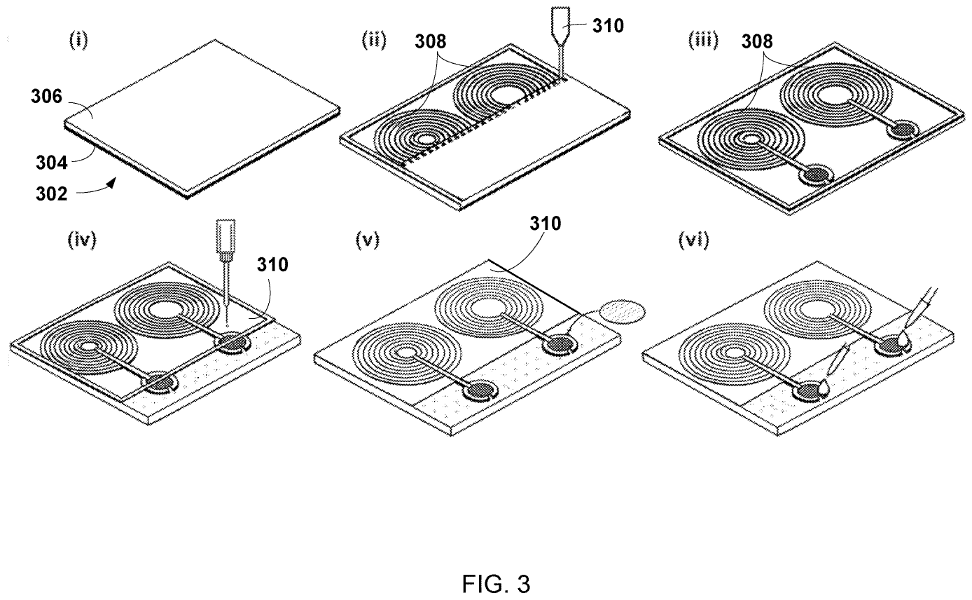

FIG. 3 illustrates an example of manufacturing the sensor platform. A metalized stock 302 (also referred to metalized substrate) having a non-conductive substrate 304 with a metalized surface 306 may be provided (i). In a possible embodiment validated according to experimentation described herein, the metalized substrate 302 was a polyethylene terephthalate (PET) substrate having a thickness of 254 μm with a 100 nm thin aluminum (Al) coated layer. However, other thicknesses and materials for the substrate and conductive coating are possible.

The fabrication may further involve etching the metalized surface to form the LC resonator-based sensors 308 (ii-iii). The surface may be etched using, for example a laser 310. In a possible embodiment validated according to experimentation described herein, the Al-metalized PET sheet was etched using a Universal Laser PLS6MW laser engraver (Scottsdale, AZ, USA) with a fiber source (1.06 μm) operating at a laser power of 16 W and a scanning speed of 1.2 m/s. Through this laser etching process, the pH and XOD LC tank sensors were patterned in a one-step laser etching process with a 2 cm gap between them to minimize electrical coupling.

The antennas of each of the LC resonator-based sensors 308 may include a corresponding spiral inductor. The number of turns for each corresponding spiral inductor may vary to differentiate the resonant characteristics of the LC resonator-based sensors. In a possible embodiment validated according to experimentation described herein, the planar spiral coils may be incorporated with IDE electrodes to detect changes in pH consisted of 10 turns, with a width of 1 mm and a gap of 2 mm between adjacent turns. The XOD sensor's spiral coil had 8 turns to ensure a distinct resonance frequency from the pH sensor. After the laser patterning process, the sensors were cleaned with isopropyl alcohol (IPA) and air-dried, followed by plasma treatment.

A passivation layer 310 may be added to the antenna of each of the LC-based resonator sensors (iv). In a possible embodiment validated according to experimentation described herein, the coils on the LC tank sensors may be passivated using heat-cured RTV silicone.

The fabrication may further involve applying a corresponding stimuli responsive coating 312 on the capacitor of each of the LC resonator-based sensors (v-vi). In a possible embodiment validated according to experimentation described herein, the IDE electrodes of a first LC-based resonator sensor used as a pH sensor were then coated with the Eudragit S100 polymer solution using an automatic dispenser (Nordson GVPlus) and dried under ambient conditions for 24 hours to form a 250 μm coating on the IDE surface. The IDE of a second LC-based resonator sensor used as a of the XOD sensor was coated with the optimized HX-infused filter paper and Eudragit EPO polymer composition and dried overnight under ambient conditions.

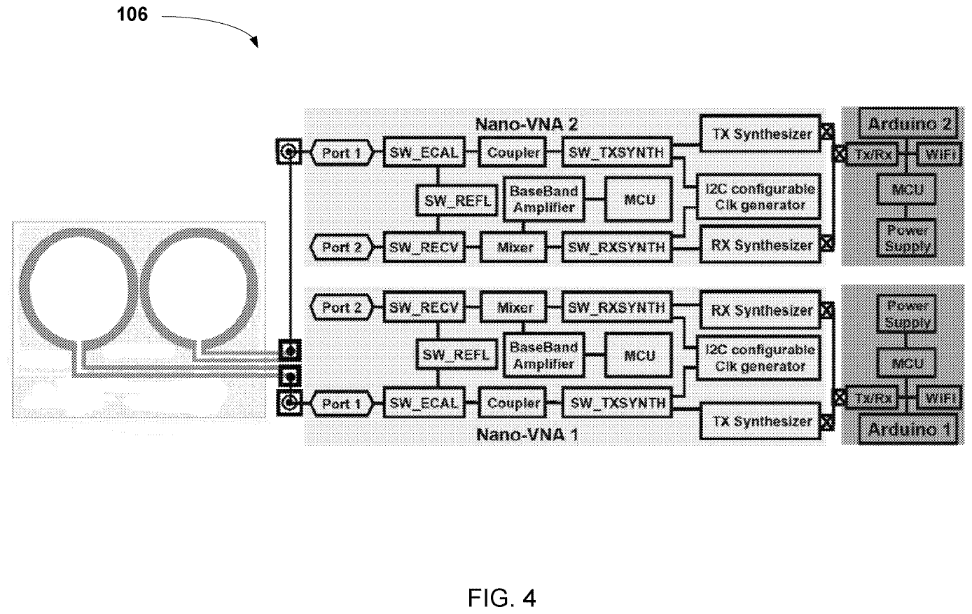

FIG. 4 illustrates an example of a wireless reader device 106 for interrogating one or more LC resonator-based sensor tags. In various embodiments, the wireless reader device 106 may be configured to automatically obtain and record sensor readings, thereby facilitating continuous or periodic monitoring in healthcare and other applications.

The wireless reader device 106 may include one or more reader antennas 108 that are positionable proximate to the sensor platform described herein (e.g., adjacent an exterior surface of a urine bag) to enable non-contact coupling to the LC resonator-based sensor(s). In some embodiments, the reader antenna 108 comprises a loop, coil, planar spiral, or other near-field antenna geometry, and may be detachable, repositionable, or temporarily secured (e.g., via adhesive, hook-and-loop fasteners, clips, or other attachment mechanisms). In some examples, the reader antenna(s) 108 may be sized and tuned to operate over a frequency range that includes expected resonant frequencies of the LC resonator-based sensor(s).

The wireless reader device 106 may further include interrogation electronics configured to generate an excitation signal and receive a response signal associated with the LC resonator-based sensor(s). In various embodiments, such interrogation electronics may include, by way of example and not limitation, a network analyzer (including a vector network analyzer), an impedance analyzer, a swept-frequency transceiver, a software-defined radio (SDR), or other circuitry capable of measuring one or more resonance characteristics (e.g., resonant frequency, peak amplitude, peak bandwidth/quality factor, phase, or reflection coefficient). The reader device 106 may include a processing device (e.g., a microcontroller, microprocessor, FPGA, system-on-chip, or similar controller) configured to control the interrogation, to process received measurements, and to output sensor data locally and/or remotely.

In one non-limiting example implementation, the reader device 106 may be designed in a modular fashion and may include two microcontrollers (e.g., Arduino MKR WiFi 1010 microcontrollers), each interfaced with a portable nano vector network analyzer (VNA) (e.g., NanoVNA-H). The VNA may include a two-port transmission/reflection architecture with an RF signal generator, directional coupler, and RF receiver, and may be configured to support a frequency sweep over a range sufficient to capture resonance behavior of the LC resonator-based sensor(s). By way of example, the VNA may utilize multiple signal sources for different frequency ranges and may provide a selected output power. The RF receiver may include a mixer and baseband amplification circuitry to down-convert received signals to an intermediate frequency (IF), and the IF may be digitized (e.g., by an ADC) for digital extraction of phase and magnitude information. In this example, two VNAs may be coupled to two reader antennas (e.g., circular reader antennas having a diameter of about 12 cm) via connectors (e.g., SMA connectors) to measure reflection coefficient characteristics (e.g., S₁₁) associated with respective LC resonator-based sensors. In some examples, the reader antenna design (including size, geometry, and operating range, such as about 50 MHz to 400 MHz) may be optimized using an electromagnetic simulation tool (e.g., HFSS). For stable and continuous measurements, the reader antenna(s) may be secured to an exterior surface of the urine bag (e.g., using hook-and-loop attachments).

In various embodiments, the reader device 106 may communicate measured data using one or more wired or wireless interfaces. For example, the reader device may transmit measurement data to a local display, a bedside monitor, a mobile device, a workstation, or a remote server using Wi-Fi, Bluetooth, cellular, Ethernet, or other suitable protocols. In one non-limiting example, reflection coefficient data (e.g., S₁₁) may be transferred and displayed in a graphical user interface (GUI) (e.g., a web-based GUI) via a network stack (e.g., TCP/IP) using a Wi-Fi radio compatible with an 802.11 network.

The components of the reader device 106 may be assembled in a protective enclosure suitable for clinical environments. In one non-limiting example, the electronics may be enclosed in a dustproof and waterproof casing (e.g., an IP65 enclosure) having dimensions on the order of tens of centimeters (e.g., about 20 cm×10 cm×7 cm).

The system may be implemented in many ways. For example, in some embodiments, the reader device 106 may utilize a single interrogation instrument and a single processing device to interrogate multiple sensor tags, including tags configured with distinct resonant frequencies. In such configurations, the reader antenna system may be designed to minimize cross-coupling between sensor tags. For instance, the reader antenna may be engineered to support dual-resonance coupling such that signals associated with two distinct LC resonators can be independently excited and received with reduced mutual interference. This may be achieved, for example, by spatial or geometric separation of coupling regions, by frequency-selective coupling paths tuned to different resonant frequencies, and/or by electromagnetic isolation techniques configured to reduce overlap between responses of different sensor tags. Although such integration can reduce overall device complexity and cost while preserving multi-sensor functionality, it may involve additional attention to antenna design and isolation to reduce interference between channels.

A second action may be said to be “in response to” a first action independent of whether the second action results directly or indirectly from the first action. The second action may occur at a substantially later time than the first action and still be in response to the first action. Similarly, the second action may be said to be in response to the first action even if intervening actions take place between the first action and the second action, and even if one or more of the intervening actions directly cause the second action to be performed. For example, a second action may be in response to a first action if the first action sets a flag and a third action later initiates the second action whenever the flag is set.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations.

The appendix attached hereto includes additional non-limiting embodiments, optimizations, and experimental validation. The appendix is incorporated by reference in its entirety.

What is claimed is:

1. A system, comprising,
a urine bag;
an inductor-capacitor (LC) resonator-based sensor disposed in the urine bag, the LC resonator-based sensor comprising an antenna connected to a capacitor with a stimuli-responsive coating disposed on the capacitor,
wherein the stimuli-responsive coating dissolves in response to presence of a stimuli in the urine thereby changing the capacitance of the capacitor and altering the resonance characteristics of the LC resonator-based sensor.

2. The system of claim 1, further comprising a sheet disposed in the urine bag, wherein the LC resonator is formed on the sheet.

3. The system of claim 1, wherein the sheet is adhered to a sidewall of the urine bag.

4. The system of claim 1, wherein a sidewall of the urine bag comprises the LC resonator-based sensor.

5. The system of claim 1, wherein the LC resonator-based sensor includes a plurality of LC resonator-based sensors, wherein the stimuli-responsive coating on each of the LC resonator-based sensors is responsive to a different stimuli.

6. The system of claim 5, wherein each of the antennas emit a different resonant frequency.

7. The system of claim 1, wherein the antenna comprises a coil inductor.

8. The system of claim 1, wherein the capacitor comprises interdigitated electrodes.

9. The system of claim 1, further comprising a wireless reader device including a reader antenna, wherein the wireless reader device is configured to wirelessly interrogate the LC resonator-based sensor via inductive coupling between the reader antenna and the antenna of the LC resonator-based sensor.

10. A system, comprising:
a plurality of inductor-capacitor (LC) resonator-based sensors formed on a flexible sheet, each of the LC resonator-based sensors comprising an antenna connected to a capacitor with a stimuli-responsive coating disposed on the capacitor,
wherein the stimuli-responsive coating dissolves in response to presence of a stimuli in the urine thereby changing the capacitance of the capacitor and altering the resonance characteristics of the LC resonator-based sensor,
wherein the stimuli-responsive coating on each of the LC-resonator based sensors is responsive to a different stimuli, and
wherein the antenna of each of the LC resonator-based sensors have a different resonant frequency.

11. The system of claim 10, wherein the antenna for each of the LC resonator-based sensors comprises a spiral coil and the capacitor for each of the LC resonator-based sensors comprises an interdigitated capacitor.

12. The system of claim 10, wherein spiral coil for each of the L resonator-based sensors has a different number of windings.

13. The system of claim 10, wherein the stimuli responsive coating a first one of the LC resonator-based sensors dissolves when exposed to fluid having a pH higher than a threshold value.

14. The system of claim 10, wherein the stimuli responsive coating of at least one of the LC resonator-based sensors dissolves when exposed to fluid having a pH higher than a threshold value.

15. The system of claim 10, wherein the stimuli responsive coating of at least one of the LC resonator-based sensors dissolves when exposed to fluid having a xanthine oxidase (XOD) concentration higher than a threshold value.

16. The system of claim 10, wherein the flexible sheet is a urine bag.

17. The system of claim 10, wherein the flexible sheet is rollable and configured to be inserted into a urine bag.

18. A method, comprising:
providing a non-conductive substrate with a metalized surface;
etching the metalized surface to form a plurality of LC resonator-based sensors, each of the LC resonator-based sensors comprising an antenna connected to a capacitor; and
applying a corresponding stimuli responsive coating on the capacitor of each of the LC resonator-based sensors.

19. The method of claim 18, wherein the antenna comprises a coil inductor and the capacitor comprises interdigitated electrodes.

20. The method of claim 18, wherein the antenna of each of the LC resonator-based sensors includes a spiral inductor, wherein the spiral inductor of each of the LC resonator-based sensors has a different number of windings.

21. The method of claim 18, wherein the corresponding stimuli responsive coating for each of the LC resonator-based sensors is different.

22. The method of claim 18, applying a passivation layer on top of the antenna of each of the LC resonator-based sensors.
